(19) United States
(12) Patent Application Publication
(10) Pub. No.: US 2017/0138636 A1
(43) Pub. Date: May 18, 2017

(54) SOLAR LENS PANEL

(71) Applicant: SUN ALGAE Technology S.R.L., Bologna (IT)

(72) Inventors: Joachim GRILL, Wien (AT); Roland RIEPL, Wels (AT)

(73) Assignee: SUN ALGAE Technology S.R.L., Bologna (IT)

(21) Appl. No.: 15/319,443

(22) PCT Filed: Jun. 18, 2015
(86) PCT No.: PCT/AT2015/050152

§ 371 Date: Dec. 16, 2016

(30) Foreign Application Priority Data
Jun. 18, 2014 (AT) A 50427/2014

(51) Int. Cl. F24J 2/06; C12M 1/12; G02B 19/00; F24J 2/08; F21S 11/00; C12M 1/00; C12M 3/00

(52) U.S. Cl. CPC F24J 2/067; C12M 21/02; C12M 31/06; C12M 23/22; C12M 23/38; C12M 43/08; C12M 25/06; C12M 25/02; C12M 23/48; F24J 2/085; F21S 11/002; G02B 19/0028; G02B 19/0042; G02B 3/08

(57) ABSTRACT

The invention relates to a solar lens panel (2) comprising a number of light collecting elements (2) placed next to one another and a number of light guides (4) corresponding to the number of light collecting elements (3); one light guide (4) including a light incident surface (5) is assigned to each light collecting element (3), and each light guide (4) is retained in a holding element (14) at a distance (8) from the light collecting elements (3); said distance (8) between the light collecting elements (3) and the light incident surfaces (5) of the light guides (4) corresponds at least approximately to the focal length of the light collecting elements (3). The light incident surfaces (5) of the light guides (4) are located inside the holding elements (14).

The invention relates to a solar lens panel comprising a number of light collecting elements placed next to one another in a plane and a number of light guides corresponding to the number of light collecting elements, wherein one light guide including a light incident surface is assigned to each light collecting element, and each light guide is retained in a holding element at a distance from the light collecting elements, wherein the distance between the light collecting elements and the light incident surfaces of the light guides corresponds at least approximately to the focal length of the light collecting elements and the area between the light incident surfaces of the light guides and the light collecting elements is preferably free of light-deflecting optical elements and preferably there is no direct mechanical connection between the light collecting elements and the light guides. Furthermore, the invention relates to a lighting system comprising at least one light supplying element and a biomass cultivating facility comprising at least one tank for holding the biomass and at least one lighting system.

In addition to the standard applications of solar power for the provision of hot water and for producing electricity it is already known from the prior art to use solar power for lighting buildings and for the production of biomass, in particular algae.

Thus for example DE 197 05 046 A1 describes a device for using solar power with a light capturing element for capturing and concentrating electromagnetic radiation from the sun, a light transport element for transporting the captured and concentrated electromagnetic radiation and a light distributor for emitting the transported electromagnetic radiation. Said device is used for illuminating buildings or closed spaces, heating buildings or closed spaces, displaying information in buildings or closed spaces, such as the cover of a solar cell, for attracting insects, for shaping watch glass or for illuminating organisms such as algae or plants.

DE 10 2007 018 675 A1 describes a biomass cultivating facility with a container for holding biomass-containing aqueous solution, with at least one light guide directed into the container for supplying light energy to the biomass-containing aqueous solution, and with a controllable light guide, which is coupled to the light guide for the optional supply of light to selected areas of the container, wherein the container is divided into segments which each have light emission surfaces which can be coupled optionally to the light guide via the light distributor, the light guide is coupled to a unit for capturing sunlight and directing the captured solar energy into the light guide and a control unit is provided for controlling the light distribution, which is set up to distribute the light output available in the light guide to the light radiation surface, such that there is an additional supply of an additional light radiation surface, when the at least one light radiation surface supplied with light output from the light guide is supplied with a lighting output required for the nominal growth in mass of the biomass and there is additional light output in order to also supply the additional light radiation surface also with a degree of lighting necessary for the nominal increase in mass of the biomass, and in that at further light radiation surfaces it is switched off such that as a function of the cumulated lighting period of a segment a predefined minimum period of cumulated dark phases is achieved.

An essential element in this type of use of solar power is the optical system, by means of which the sunlight is directed into the light guide.

From EP 2 189 711 A2 a coupling unit is known for coupling sunlight into a fiber made from a block of light-conductive material, comprising a surface for the light input, a light guide with an input side and an opposite side which displays light entering on the input side onto the opposite side, and a fiber coupling for coupling the device to the fiber. Said coupling unit is connected directly to the optical lens. By means of said coupling unit a maintenance-free and robust assembly is achieved for coupling light into a fiber. In addition, in this way the manufacturing costs and the operating costs of solar lens panel for lighting are reduced. The disadvantage of this is however that the optical assemblies consisting of the lens and the coupling unit during the series production of such solar lens panels still have high production costs, as a relatively large amount of material is required and the production time of said system is also relatively long.

However, in the prior art two-part optical assemblies have already been described, in which the light guides are not coupled directly to the optical lenses. Such a system is known for example from U.S. Pat. No. 6,299,317 B1. According to the latter light is captured via Fresnel lenses and projected into a light guide, whereby there is no direct connection between the Fresnel lenses and the light guides. However, said system has conical, parabolic concentrators between the Fresnel lenses and the light guides as additional optical elements, in order to concentrate the incidental light bundle on the light guides. By means of said secondary lens and the more complex structure associated therewith the system gets more expensive.

The underlying objective of the present invention is to create a solar lens panel of the aforementioned kind which can be produced inexpensively in series and with a high degree of automation, so that such solar lens panels can be used more widely for lighting, in particular in the production of biomass and in this way energy costs and greenhouse gases can be reduced.

Said objective of the invention is achieved in the aforementioned solar lens panel in that the light incident surfaces of the light guides are arranged inside the holding elements. Furthermore, the objective of the invention is achieved by the aforementioned lighting system which comprises such a solar lens panel. In addition, the objective of the invention is achieved by the aforementioned biomass cultivating facility which comprises such a lighting system.

It is an advantage that the solar lens panel comprises only a single optical element for each light guide, namely the light collecting element. In this way it is possible to simplify the structure of the solar lens panel, in particular it is no longer necessary to adjust the primary lens, i.e. the light collecting elements, to a secondary lens, as used in the prior art. This is associated with the disadvantage that the precision of the distance adjustment between the primary lens and the light guides has to be greater to meet higher requirements, however the advantage of the smaller number of individual components outweighs this disadvantage. In addition, by avoiding having a secondary lens the production costs are reduced by an amount which is greater than the additional effort of precisely adjusting the distance between the light collecting elements and the light guides. It is thus also an advantage that it is simpler to produce the individual components in serial production for example by means of an injection molding method, whereby it is also possible to reduce costs accordingly.

The light guides are arranged respectively in a holding element. In this way the light guides can be held more effectively in the required position, whereby the adjustment of the light collecting elements can be achieved more easily in relation to the light guides.

It is also the case that the light incident surfaces of the light guides are arranged in the holding elements. In this way overheating protection is provided in the region of the light incident surface. Impurities in this area can of course result in the absorption of energy so that the holding elements get hot. Since the focus on the light incident surfaces of the light guides is inside the holding elements, the focused light beam is still relatively wide on entering the holding element, so that the energy is not sufficient to heat the holding elements to the extent that the holding elements get damaged and thus the light guides lose their precise relative position relative to the light collecting elements.

A further improvement relating to the degree of automation of the production of the solar lens panel and thus also to a reduction in costs can be achieved if a plurality of light collecting elements form a one-piece light collecting element module. In this way however also the adjustment of the distance between the light collecting elements and the light incident surfaces of the light guide is simplified, as not each individual spacing has to be adjusted separately. This thus results in a further simplification of the assembly of the solar lens panel and thereby a further reduction of the production costs.

Preferably, the light collecting elements are in the form of Fresnel lenses. In this way it is possible to achieve a significant reduction of the installation space required by the solar lens panel so that the latter can be designed to be flatter.

According to another embodiment variant of the solar lens panel it is possible that a plurality of holding elements are arranged in an, in particular, one-piece holding element module. In this way a further reduction of the production costs can be achieved by means of a greater degree of automation and a simpler assembly of the solar lens panel. Furthermore, in this way it is also possible that the plurality of holding elements can be arranged more simply in a common plane.

For the more simple adjustment of the focal point of the light collecting elements to the light incident surfaces of the light guides it is possible to arrange spacers between the light collecting element module(s) and the holding element module(s), in particular the light collecting element module(s) are connected via the spacers to the holding element module or modules.

Preferably, the light collecting elements and the holding elements are arranged in a common frame, whereby the solar lens panel can be designed to be more resistant to environmental influences.

It is also possible for the light collecting elements to be covered with a transparent cover. In this way it is possible to protect the light collecting element and the underlying light guide more effectively from environmental influences. In particular, in this way it is possible to prevent more effectively the holding element from getting dirty, whereby it is also possible to prevent (more effectively) the overheating of said holding elements from energy absorption. It is thus possible that adhesions within the solar lens panel can be provided less expensively using less material, for example the adhesion of the light guides to the holding elements. Similarly, in this way the adhesions or the materials inside the solar lens panel can be better protected from the effects of UV-radiation.

For the same reason it is possible that the light collecting elements and the holding elements are arranged in a closed system, whereby preferably the closed system comprises the frame, the transparent cover and a base plate, wherein at least one membrane is arranged on the frame and/or on the transparent cover and/or on the base plate. By means of the latter embodiment the solar lens panel can be closed completely so that the solar lens panel is prevented from getting dirty on the inside or this can be reduced considerably, wherein by means of the at least one membrane there can be an exchange of air and water between the inner chamber of the solar lens panel and the surrounding atmosphere.

The base plate is preferably formed by the holding element module or modules, as in this way it is possible to achieve a further simplification of the structure of the solar lens panel, whereby material costs can be reduced.

For a better understanding of the invention the latter is explained in more detail with reference to the following Figures.

In a simplified, schematic representation:

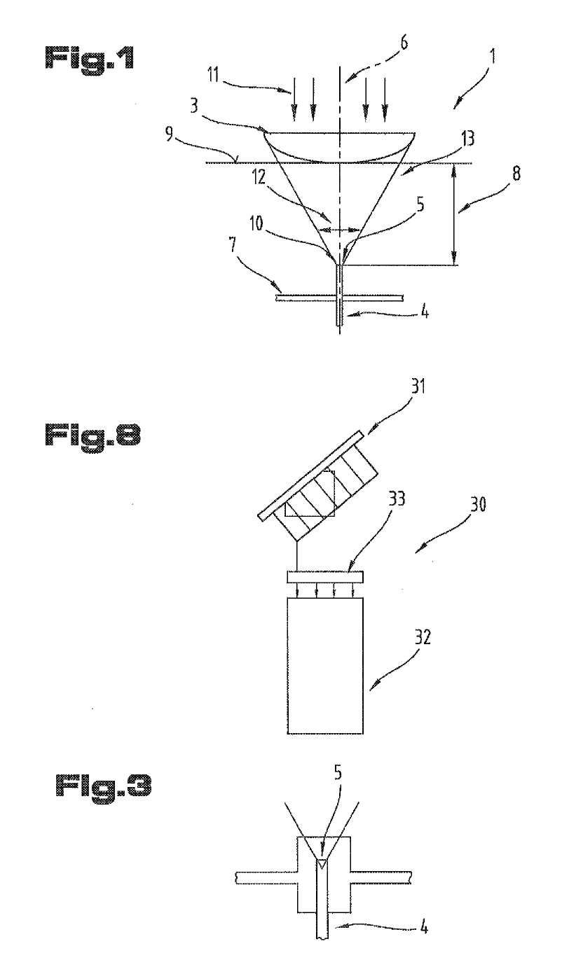

FIG. 1 shows an optical assembly for a solar lens panel;

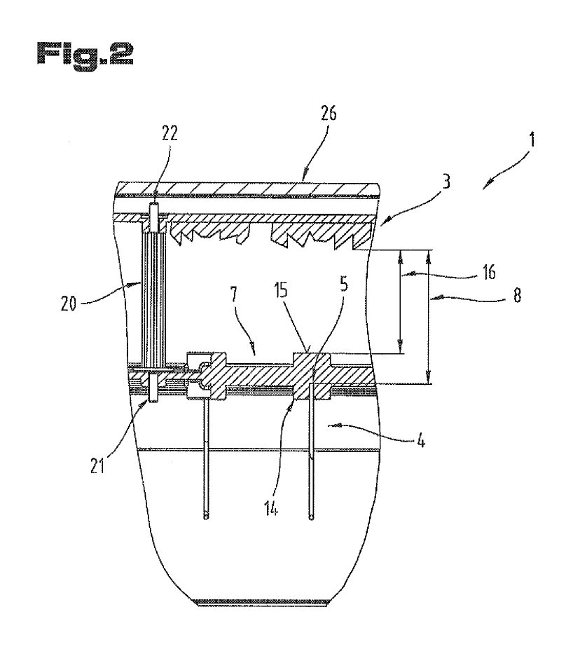

FIG. 2 shows an optical assembly for a solar lens panel;

FIG. 3 shows a detail of an optical assembly;

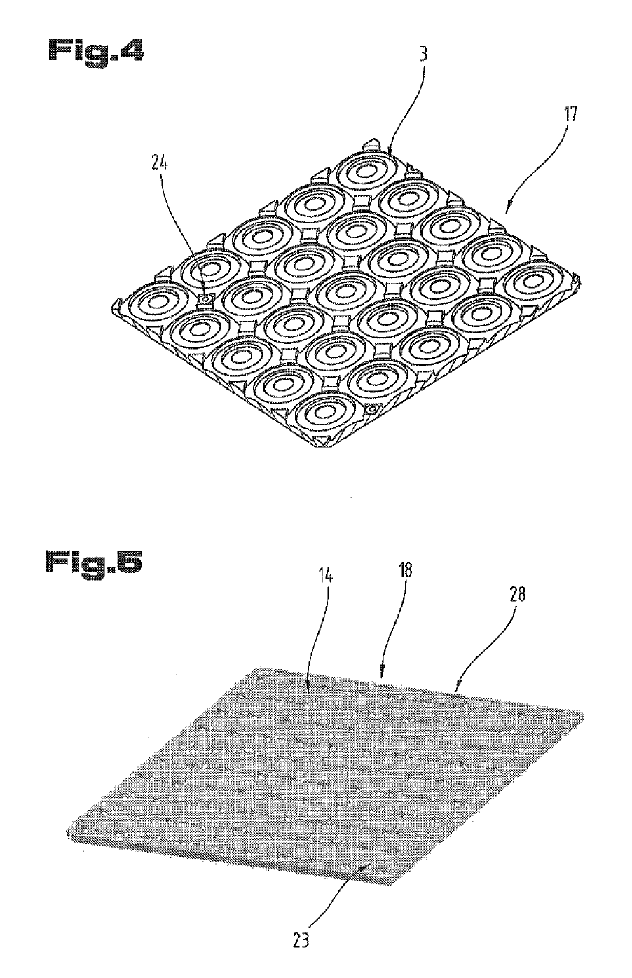

FIG. 4 shows a light collecting element module in perspective;

FIG. 5 shows a base plate in perspective;

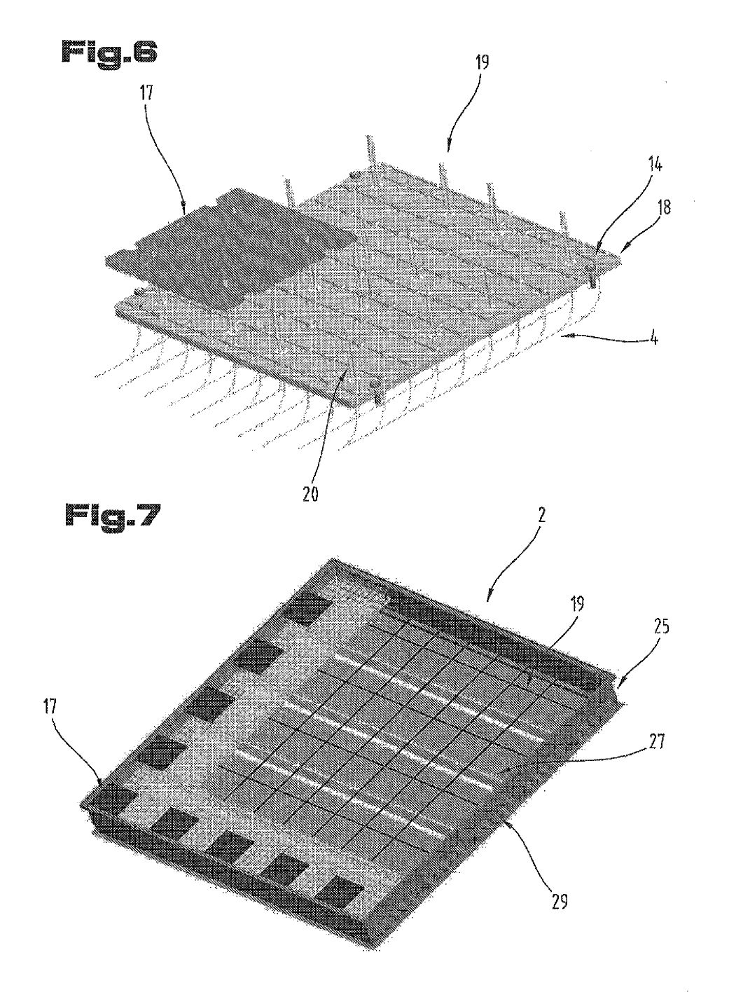

FIG. 6 shows a partly assembled solar lens panel in perspective;

FIG. 7 shows a solar lens panel in perspective;

FIG. 8 shows a cut-out of a biomass cultivating facility.

First of all, it should be noted that in the variously described exemplary embodiments the same parts have been given the same reference numerals and the same component names, whereby the disclosures contained throughout the entire description can be applied to the same parts with the same reference numerals and same component names. Also details relating to position used in the description, such as e.g. top, bottom, side etc. relate to the currently described and represented figure and in case of a change in position should be adjusted to the new position.

FIG. 1 shows a simplified optical assembly 1 for a solar lens panel 2 shown in FIG. 7.

Said optical assembly consists of a light collecting element 3 and a light guide 4.

The light collecting element 3 is designed as a collective lens. In the shown embodiment variant the collective lens is a so-called planar convex lens.

The light guide 4 is arranged underneath the light collecting element 3. It comprises a light incident surface 5 which faces the light collecting element 3 and is at right angles to an optical axis 6 of the light collecting element 3. Preferably, the optical axis runs through the central point of the light incident surface 5 of the light guide 4 designed to have an at least approximately circular cross-section.

The light guide 4 is held at a distance 8 by a mount 7, which is designed to be plate-like for example, wherein the light guides 4 can be guided through a bore in the mount 7. The distance 8 between the light collecting element 3 and the light incident surface 5 of the light guide 4 corresponds at least approximately to the focal distance of the light collecting element 3. The focal distance is defined here as the distance of a main plane 9 of the light collecting element 3 from a focal point 10 of the light collecting element 3. In other words, thus incidental light 11 is focused by the light collecting element 3 at least approximately or precisely onto the light incident surface 5 of the light guide 4.

Within the meaning of the invention the term “at least approximately to the light incident surface 5” means that the diameter 12 of a light cone 13 on the light incident surface 5 of the light guide 4 is not more than 20% greater, in particular not more than 10% greater, than the diameter of the light incident surface 5 of the light guide 4.

The diameter 12 of the light cone 13 can however also correspond to the diameter of the light incident surface 5 of the light guide 4 and the focal point of the light collecting element 3 can be in the light incident surface 5 of the light guide 4.

Preferably, however the focal point of the light collecting element 3 inside the light guide 4 is below the light incident surface 5, as shown in FIG. 3, so that the energy input in the region of the light incident surface 5 is lower than if the focal point were to be on the light incident surface 5 of the light guide 4. In this way the energy of the light cone is not sufficient to melt the plastic. The distance of the focal point from the light incident surface 5 can thus be a maximum of 1.5 mm (i.e. between 0 mm and 1.5 mm), in particular up to a maximum of 1 mm.

The distance 8 between the light collecting element 3 and the light incident surface 5 of the light guide 4 thus preferably corresponds to a maximum of the focal distance of the light collecting element 3 with a tolerance range of ±0.1 mm.

It should be mentioned that the terms “optical axis”, “focal distance” and “focal point” have the usual optical definitions.

Between the light collecting element 3 and the light guide 4 no further optical elements, are arranged, i.e. no secondary lens. The area between the light collecting element 3 and the light incident surface 5 of the light guide 4 is thus free of light-refracting and free of light-deflecting elements.

Furthermore, there is no direct mechanical connection between the light guide 4 and the light collecting element 3.

In other words the optical assembly 1 according to FIG. 1 consists of the light collecting element 3 and the light guide 4 spaced apart from the latter.

FIG. 2 shows an embodiment of the optical assembly 1 in cross-section, wherein for the same parts the same reference numerals and component names have been used as in the description for FIG. 1. To avoid unnecessary repetition reference is made to the detailed description above.

The optical assembly 1 is the preferred embodiment of the invention. The optical assembly 1 consists of the light collecting element 3 and the light guide 4 with the light incident surface 5.

The light collecting element 3 is preferably a Fresnel lens. As Fresnel lenses are known in principle, reference is made to the relevant prior art. However, another suitable type of lens can also be used.

The light incident surface 5 of the light guide 4 is arranged at the distance 8 from the light collecting element 3 described in more detail above.

Furthermore, the light guide 4 is held in a holding element 14, in particular is adhered to the latter, which is in turn arranged on the mount 7. It is thus also possible for the holding element 14 and the mount 7 to form a single-piece component made from one material.

In the preferred embodiment of the optical assembly 1 shown in FIG. 2 the light incident surface 5 is arranged inside the holding element 14. The distance 16 between the end surface 15 of the holding element 14 and the light collecting element 3 is thus preferably smaller than the distance 8 between the light collecting element 3 and the light incident surface 5 of the light guide 4.

For the adhesion of the light guide 4 inside the holding element 14 preferably, the holding element 14 has a recess, in particular a blind bore. The cross-section of said recess is preferably greater than the cross-section of the light guide 4 as viewed in the same direction. It is thus possible to form at least one channel between the light guide 4 and the wall of the recess, by means of which the air located in the recess can escape through the adhesive which is displaced by the insertion of the light guide 4 into the recess. In addition, by means of the displaced adhesive a seal can be achieved of the recess on the underside of the holding element 14 at which the light guide 4 exits from the holding element 14.

The recess can have for example an oval or in particular square, preferably quadratic cross-section. The light guide 4 preferably has an at least approximately circular cross-section.

For the same purpose however it is also possible to design at least one channel in the wall of the recess as a depression in the latter.

According to a not preferred embodiment variant it is possible that a transverse bore is made in the holding element 14, which bore extends from the outside into the recess. It is hereby possible that a plurality of such transverse bores are made over the height of the embedding of the light guide 4 and/or distributed around the periphery of the recess. Air can also escape through said transverse bores and by means of the escaping adhesive a seal or closure of said transverse bores can be achieved.

By avoiding air pockets between the light guide 4 and the holding element 14 it is easier to prevent change to the optical behavior of the combination of holding element 14/light guide 4 and the possible overheating of the light guide 4 in the region of the light incident surface 5.

Preferably, a UV-resistant adhesive is used as the adhesive. It is also preferable if the adhesive does not cause any optical refraction of the light passing through.

For this reason the holding element 14 is made, at least in the preferred embodiment of the invention from a light-permeable (transparent) material. However, no light deflection takes place in the holding element 14. Thus in this embodiment variant of the optical assembly 1 according to FIG. 2 no optically active element (in terms of light deflection) is arranged between the light collecting element 3 and the light incident surface 5 of the light guide 4. Furthermore, the end surface 15 of the holding element 14 is preferably designed to be shiny or high-gloss (but not reflective). In addition, the end surface 15 is preferably oriented parallel to the main plane 9 (see FIG. 1) of the light collecting element 3 and in particular also to the light incident surface 5 of the light guide 4.

It is also preferable if the holding element 14 is made from a material which has a refractive index, which differs by not more than 5%, in particular by not more than 3%, from the refractive index of the material of the light guide 4.

Preferably, there is no further light refraction in the holding element 14.

The holding element 14 can be designed for example in the form of a cylinder or square, wherein these forms should not be considered to be restrictive.

Furthermore, in this embodiment variant of the invention there is no direct connection between the light collecting element 3 and the light guide 4. In other words, the area (space) directly below the light collecting element 3 and between the light collecting element 3 and the light guide 4, with the exception of the holding element, is free of other elements or components. The space between the light collecting element 3 and the light guides 4 can thus be filled with a gas or evacuated. As explained in more detail in the following, the holding element 14 can be part of a holding element module and the light collecting element 3 can be part of a light collecting element. These two modules can be connected to one another by spacers. Said spacers are located next to the holding elements 14, i.e. within the meaning of the invention not in the area immediately below the light collecting element 3 and between the light collecting element 3 and the light guide 4 and do not have an optical function in terms of light guiding or light deflection.

The solar lens panel 2 according to the invention, as shown for example in FIG. 7 in the preferred embodiment, comprises a plurality of optical assemblies 1 (FIGS. 1 and 2). Thus the solar lens panel comprises a number of light collecting elements 3 and a number of light guides 4 corresponding to the number of said light collecting elements 3. All of the light collecting elements 3 are preferably arranged in one plane. Likewise the light incident surfaces 5 of the light guides 4 are preferably arranged in a plane. Preferably, the plane of the light incident surfaces 5 of the light guides 4 is aligned parallel to the plane of the light collecting elements 3.

FIG. 4 shows a further embodiment of the invention, by means of which the arrangement of the light collecting elements 3 in a plane can be simplified. The light collecting elements 3 are preferably Fresnel lenses.

In this embodiment variant a plurality of light collecting elements 3 form a one-piece light collecting element module 17.

In the shown embodiment variant the light collecting element module 17 comprises twenty five light collecting elements 3, which are divided into five rows and five columns.

The light collecting element module 17 can however also have more or fewer than twenty five light collecting elements 3, for example a hundred, in that the view in FIG. 4 is only given by way of example. Furthermore, the light collecting element module 17 does not necessarily need to be square (as shown), i.e. the number of rows and columns can differ from one another.

A further way of simplifying the alignment of the light incident surfaces 5 of the light guides 4 (FIGS. 1 and 2) is shown in FIG. 5, in which a further embodiment variant is shown which can be used alternatively or additionally to the embodiment variant according to FIG. 4 in the solar lens panel 2 (FIG. 7).

In this embodiment variant a plurality of holding elements 14 are arranged in an, in particular one-piece, holding element module 18. The location of the holding elements 14 in the holding element module 18 is selected so that a holding element 14—and also a light guide 4—is arranged underneath a light collecting element 3, as shown in FIGS. 1 and 4.

The holding element module 18 according to FIG. 5 comprises 100 holding elements 14 which are divided into 10 rows and 10 columns. However, it is also possible that the holding element module 18 comprises more or fewer holding elements 14, for example up to 200, and that the number of rows differs from the number of columns.

In the preferred embodiment variant however the holding element module is designed to have more holding elements 14 than the number of light collecting elements 3 of the light collecting element module 17 according to FIG. 4, so that for each holding element module 18 a plurality of light collecting element modules 17 are installed in the solar lens panel 2 as shown in FIGS. 6 and 7.

FIG. 6 shows a solar lens panel module 19 which comprises a holding element module 18 according to FIG. 5 and four collecting element modules 17 according to FIG. 4.

Furthermore, in FIG. 6 the light guides 4 are shown which are held by the holding elements 14.

FIG. 7 shows that the solar lens panel 2 can comprise a plurality of said solar lens panel modules 19, for example twenty five as shown in FIG. 7, but also more or fewer. In order to illustrate the relationship more effectively, in FIG. 7 shows the solar lens panel modules shown partly in FIG. 6. Of course, all solar lens panel modules 19 are provided completely with the light collecting elements 17.

The solar lens panel 2 is thus largely modular in structure.

In order to simplify the assembly of said solar lens panel, hi particular to adjust more easily the distance 8 (FIGS. 1 and 2) between the light collecting elements 3 and the light incident surfaces 5 of the light guides 4, according to a further embodiment variant of the solar lens panel 2 spacers 20 can be arranged between the light collecting element module or modules 17 and the holding element module or modules 18, as shown in particular in FIGS. 2 and 5. By means of said spacers 20 the light collecting element module or modules 17 can be connected to the holding element module or modules 18, for example in that the spacers 20 are adhered, welded or generally materially-bonded to the light collecting element module(s) 17 with the holding element module or modules 18.

Said spacers 20 are installed outside the area between the light collecting elements 3 and the light guides 4, so that the light collecting elements 3 and the light guides 4 are not connected to one another directly, but are connected to one another indirectly by the spacers 20 which in turn connect together the light collecting element module(s) 17 and holding element module(s) 18.

The spacers 20 are preferably designed to be rod-like and each comprise in the end sections a plug 21, 22, as shown in FIG. 2. The plugs 21 can engage in bores 23 (FIG. 5) or recesses in the holding element module 18. The plugs 22 can in turn engage in bores 24 (FIG. 4) or recesses in the collecting element module 17. To create the connection the plugs 21, 22 can simply be inserted into the bores 23, 24. In addition, the plugs 21, 22 can also be adhered or welded or generally materially-bonded to the collecting element modules 17 and/or the holding element modules 18.

By means of the spacers 20 it is possible to prevent (more effectively) the tilting of the at least one holding element module 18 relative to the at least one collecting element module 17.

The bores 24 in the holding element module 18 are arranged next to the holding elements 14, as shown in FIG. 5. In particular, they are arranged in the middle point of the surface spanned by four holding elements 14.

The bores 23 in the light collecting element module 17 are preferably arranged in the corner points between the collecting elements 3.

As shown in FIG. 6, a spacer 20 can be connected to more than one collecting element module 17. In particular, two or four collecting element modules 17 arranged next to one another can be provided with or connected by a common spacer 20, as shown in FIG. 6.

It is also possible that the solar lens panel 2 comprises only one light collecting element module 17 and/or only one holding element module 18.

As shown in FIG. 7 the light collecting elements 3 or the light collecting element modules 17 and the holding elements 14 or the holding element modules 18 can be arranged inside a common frame 25.

The frame 25 can be made for example of aluminum or an aluminum alloy. The frame 25 comprises in particular the whole arrangement of the light collecting elements 3 or the light collecting element modules 17 and the holding elements 14 or the holding element modules 18 on the outermost periphery.

Furthermore, it is also possible that the light collecting elements 3 or the light collecting element modules 17 are covered by a transparent cover 26, as shown in FIG. 2. The cover 26 can be made from a polymeric plastic or glass or from another suitable material. Preferably, the cover 26 is connected to the frame 25, in particular adhered, so that there is no direct contact between the light collecting elements 3 or the light collecting element modules 17 with the cover 26.

In order with larger area solar lens panels 2 to achieve a further support of the cover 26, support webs 27 can be arranged between the light collecting elements 3 or the light collecting element modules 17 and possibly between the holding elements 14 or the holding element modules 18, as shown in FIG. 7.

In the preferred embodiment the light collecting elements 3 or the light collecting element modules 17 and the holding elements 14 or the holding element modules 18 are arranged inside a closed system which is preferably formed by the frame 25, the transparent cover 26 and a base plate 28 (FIG. 5), wherein the base plate 28 can be formed by or can comprise one or more holding element modules 18. However, it is also possible that the base plate 28 forms a separate component on which the holding elements 14 or the holding element modules 18 can be arranged and in particular can be connected to the latter.

It is an advantage with respect to the heating and cooling of the inner chamber of said closed system due to the periodic radiation from the sun, if at least one membrane 29 is arranged on and connected to the frame and/or the transparent cover and/or the base plate, by means of which at least one opening through the frame 25 and/or the transparent cover 26 and or the base plate 28 is closed. Preferably, a plurality of openings are formed which are closed respectively by a membrane 29, wherein one membrane 29 can also close a plurality of open-lugs. An exchange of gas (air exchange) and removal of water vapor from the inner chamber of the closed system can be performed via the membrane(s) 29.

The light collecting elements 3 or the light collecting element modules 4 and/or the holding elements 14 or the holding element modules 18 can be made at least partly, preferably fully from a polymeric, synthetic plastic. The plastic used can be for example PMMA or PC or generally a highly transparent plastic.

The light guides 4 can be made of glass or from a polymeric, synthetic plastic, for example PMMA.

Preferably, the conductors 4 and the holding element module or modules 18 are made from the same material.

The holding elements 14 or the holding element modules 18 and/or the light collecting elements 3 or the light collecting element modules 17 are preferably produced by means of an injection stamping method. As injection stamping is a well-known process, reference is made to the relevant literature.

The holding elements 14 or the holding element modules 18 and/or the light collecting elements 3 or the light collecting element modules 17 can also be produced by injection molding or by means of machining methods, e.g. milling.

It is possible by means of this method to produce the holding elements 14 or the holding element modules 18 and/or the light collecting elements 3 or the light collecting element modules 17 to have extremely low tolerances (in particular a maximum of ±0.05 mm).

The solar lens panel 2 is used in particular for a lighting system, for example in order in this way to direct sunlight into the inside of a building.

Preferably, the lighting system is used in a biomass cultivating facility 30, as shown schematically in FIG. 8. The biomass cultivating facility 30 comprises at least one light supplying element 31, which is part of the lighting system and is formed by the solar lens panel 2.

Furthermore, the biomass cultivating facility 30 comprises at least one tank 32, in which the biomass, e.g. algae, is cultivated.

In order to achieve the distribution of the light in the tank or tanks 32 a light distributing element 33 can also be arranged between the at least one solar lens panel 2 and the at least one tank.

As such biomass cultivating facilities 30 are known in principle from the prior art, for further details reference is made to the relevant prior art.

The solar lens panel 2 can be connected to a drive device to allow it to track the moving sun.

The example embodiments show possible embodiment variants of the solar lens panel 2, whereby it should be noted at this point that also various different combinations of the individual embodiment variants are possible.

Finally, for the sake of formality it should be noted that for a better understanding of the structure of the solar lens panel 2 the latter and its components have not been shown to scale in part and have been enlarged and/or reduced in size.

List of reference numerals

| | |
|---|---|
| 1 | assembly |
| 2 | solar lens panel |
| 3 | light collecting element |
| 4 | light guides |
| 5 | light incident surface |
| 6 | axis |
| 7 | mount |
| 8 | distance |
| 9 | main plane |
| 10 | focal point |
| 11 | light |
| 12 | diameter |
| 13 | light cone |
| 14 | holding element |
| 15 | end surface |
| 16 | distance |
| 17 | light collecting element module |
| 18 | holding element module |
| 19 | solar lens panel module |
| 20 | spacer |
| 21 | plug |
| 22 | plug |
| 23 | bore |
| 24 | bore |
| 25 | frame |
| 26 | cover |
| 27 | support web |
| 28 | base plate |
| 29 | membrane |
| 30 | biomass cultivating facility |
| 31 | light supplying element |
| 32 | tank |
| 33 | light distributing element |

1: A solar lens panel (2) comprising a number of light collecting elements (3) placed next to one another, in particular in one plane, and a number of light guides (4) corresponding to the number of light collecting elements (3), wherein each light collecting element (3) is assigned with one light guide (4) including a light incident surface (5) and each light guide (4) is retained in a holding element (14) at a distance (8) from the light collecting elements (3), wherein the distance (8) between the light collecting elements (3) and the light incident surfaces (5) of the light guides (4) corresponds at least approximately to the focal length of the light collecting elements (3) and the area between the light incident surfaces (5) of the light guides (4) and the light collecting elements (3) is preferably free of light-deflecting optical elements and preferably there is no direct mechanical connection between the light collecting elements (3) and the light guides (4), wherein the light incident surfaces (5) of the light guides (4) are arranged inside the holding elements (14).

2: The solar lens panel (2) as claimed in claim 1, wherein a plurality of light collecting elements (3) form a light collecting element module (17), in particular a one-piece light collecting element module (17).

3: The solar lens panel (2) as claimed in claim 1 or 2, wherein the light collecting elements (3) are in the form of Fresnel lenses.

4: The solar lens panel (2) as claimed in claim 1, wherein a plurality of holding elements (14) are arranged in a holding element module, in particular a one-piece holding element module (18).

5: The solar lens panel (2) as claimed in claim 4, wherein between the light collecting element module or module (17) and the holding element module(s) (18) spacers (20) are arranged, in particular the light collecting element module or modules (17) are connected via the spacers (20) to the holding element module or modules (18).

6: The solar lens panel (2) as claimed in claim 1, wherein the light collecting elements (3) and the holding elements (14) are arranged in a common frame (25).

7: The solar lens panel (2) as claimed in claim 1, wherein the light collecting elements (3) are covered by a transparent cover (26).

8: The solar lens panel (2) as claimed in claim 1, wherein the light collecting elements (3) and the holding elements (14) are arranged in a closed system.

9: The solar lens panel (2) as claimed in claim 8, wherein the closed system comprises the frame (25), the transparent cover (26) and a base plate (28), wherein at least one membrane (29) is arranged on the frame (25) and/or on the transparent cover (26) and/or on the base plate (28).

10: The solar lens panel (2) as claimed in claim 9, wherein the base plate (28) is formed by the holding element module or modules (18).

11: A lighting system comprising at least one light supplying element, wherein the at least one light supplying element is formed by a solar lens panel (2) as claimed in claim 1.

12: A biomass cultivating facility (30) comprising at least one tank (32) for holding the biomass and at least one lighting system, which is operatively connected to the tank (32), wherein the at least one lighting system is designed according to claim 11.
